(19) United States
(12) Patent Application Publication
(10) Pub. No.: US 2009/0203147 A1
(43) Pub. Date: Aug. 13, 2009

(54) BLOTTING DETECTION METHOD

(76) Inventor: Junichi KATADA, Kanagawa (JP)

Correspondence Address: BIRCH STEWART KOLASCH & BIRCH, PO BOX 747, FALLS CHURCH, VA, 22040-0747, US

(21) Appl. No.: 12/369,518

(22) Filed: Feb. 11, 2009

(30) Foreign Application Priority Data
Feb. 12, 2008 (JP) 2008-029985

(51) Int. Cl. G01N 33/20

(52) U.S. Cl. 436/80

(57) ABSTRACT

It is an object of the present invention to provide a blotting detection method capable of quick detection of a very tiny amount of an analysis target. The present invention provides a blotting detection method which comprises moving an analyte held on a first carrier with a developing solution, and adsorbing the analyte onto a second carrier, wherein the analyte is labeled with metal fine particle and is detected by sensitization with use of a silver-containing compound and a reducing agent for silver ion, and the labeling substance having a size of not less than 1 μm and not more than 20 μm in the average particle size at the time of detection is detected.

### TECHNICAL FIELD

The present invention relates to a blotting method for speeding up the detection of a target substance.

### BACKGROUND ART

Blotting is a method for detecting a protein or a nucleic acid existing in a test solution by: transferring the protein or nucleic acid held on a first carrier such as an agarose gel in which the test solution has been electrophoresed, onto another second carrier bindable thereto through adsorption; and by labeling the protein or nucleic acid with a labeling substance (a fluorescent substance, an enzyme, a fine metal particle, or the like) that is specifically bindable to the protein or nucleic acid. With regard to such a blotting detection method using a gold label, a technique for highly sensitive detection through silver sensitization of the gold label has been known, and such a kit (Silver Enhancing Kit) is commercially available by BBI and other companies. Upon the detection through silver sensitization with use of this kit, the silver sensitization process takes a time of 5 minutes to 30 minutes.

### DISCLOSURE OF THE INVENTION

As described above, with regard to the blotting detection method using a gold label, a technique for highly sensitive detection through silver sensitization of the gold label has been known, and such a kit is commercially available by BBI and other companies. However, there has been a problem in that the silver sensitization process takes a time of 5 minutes to 30 minutes upon the detection through silver sensitization with use of this kit. In addition, although the detection sensitivity depends on the post-sensitization visibility (absorbance), the sensitization using a conventional kit is insufficient in the sensitivity and has been incapable of detecting a very tiny amount of protein or nucleic acid in a test solution. It is an object of the present invention to provide a blotting detection method capable of quick detection of a very tiny amount of an analysis target which has been impossible to analyze with conventional blotting methods.

In the present invention, it was found that an analyte can be quickly detected by: labeling the analyte with gold fine particle; detecting it through sensitization with use of a silver-containing compound and a reducing agent for silver ion; and detecting the labeling substance having a size of not less than 1 μm and not more than 20 μm in the average particle size at the time of detection. This has led to the completion of the present invention.

The present invention provides a blotting detection method which comprises moving an analyte held on a first carrier with a developing solution, and adsorbing the analyte onto a second carrier, wherein the analyte is labeled with metal fine particle and is detected by sensitization with use of a silver-containing compound and a reducing agent for silver ion, and the labeling substance having a size of not less than 1 μm and not more than 20 μm in the average particle size at the time of detection is detected.

Preferably, the second carrier is porous.

Preferably, the analyte is a protein or a nucleic acid.

Preferably, the metal fine particle is a gold fine particle.

Preferably, the time for the sensitization reaction with use of the silver-containing compound or the reducing agent for silver ion is within five minutes.

Preferably, the time for the sensitization reaction is within two minutes.

Preferably, the silver-containing compound is silver nitrate.

Preferably, the reducing agent for silver ion is Fe⁺².

The present invention, as compared to conventional techniques, is capable of shortening the time for silver sensitization, and is capable of improving the minimum detection sensitivity for a protein, by using a sensitization solution which can greatly enlarge the post-sensitization size of silver particles (particle diameter of 1 μm or more) in a short time.

### BRIEF DESCRIPTION OF THE DRAWING

FIG. 1 shows experimental results of the evaluation of detection sensitivity.

### BEST MODE FOR CARRYING OUT THE INVENTION

### 1. Blotting Detection Method

The blotting detection method of the present invention comprises moving an analyte held on a first carrier with a developing solution, and adsorbing the analyte onto a second carrier, wherein the analyte is labeled with a fine metal particle-labeled substance that is bindable to the analyte, and is detected by sensitization with use of a silver-containing compound and a reducing agent for silver ion, and the labeling substance having a size of not less than 1 μm and not more than 20 μm in the average particle size at the time of detection is detected. The average particle size at the time of detection is preferably not less than 3 μm and not more than 20 μm, and more preferably not less than 5 μm and not more than 20 μm.

As to the first carrier, a carrier for general use in electrophoresis of proteins and nucleic acids, such as a polyacrylamide gel and an agarose gel, can be employed.

As to the second carrier, a porous carrier is preferred. In particular, a nitrocellulose membrane, a cellulose membrane, an acetyl cellulose membrane, a polyvinylidene difluoride (PVDF) membrane, a polysulfone membrane, a polyether sulfone membrane, a nylon membrane, glass fibers, a nonwoven fabric, a cloth, or the like, can be employed.

As to the developing solution, any general buffer for use in biological experiments, such as a Tris buffer and a glycine buffer, may be employed. The type, pH, and salt strength of the buffer may be adjusted for the substance to be detected. In addition, for the purpose of controlling the electric charge or the shape of the substance to be detected for performing the development, those containing a surfactant component such as SDS may also be employed. Also, a non-water solvent such as methanol may also be contained therein.

### 2. Test Sample

The test sample that can be analyzed by the blotting detection method of the present invention is not specifically limited as long as it is a sample that possibly contains an analysis target such as a protein and a nucleic acid. Examples thereof can include biologically-derived liquids (such as blood, cell lysate, secretion, culture solution of microbes, and other body fluids), and purified matters thereof.

### 3. Pretreatment of Test Sample

In the blotting detection method of the present invention, the test sample may be used in an intact form, in a form of an extract obtained by extracting the test sample with a suitable extraction solvent, in a form of a diluted solution obtained by diluting the extract with a suitable diluent, or in a form of a concentrate obtained by concentrating the extract by a suitable method. The extraction solvent to be employed may be a solvent for use in ordinary immunological analysis methods (for example, water, physiological saline, or a buffer solution) or a water-miscible organic solvent in which the antigen-antibody reaction can be directly carried out through dilution with a solvent as mentioned above.

### 4. Detection Label

As to the detection label, metal fine particles are used. Examples thereof can include a gold colloid. The average particle diameter of the gold colloid is preferably about 1 nm to 500 nm, and more preferably 1 nm to 50 nm. The substance bindable to the analyte can be labeled with fine metal particles according to a conventionally known method (for example, The Journal of Histochemistry and Cytochemistry, Vol. 30, No. 7, pp. 691-696 (1982)). For example, if the analyte-bindable substance is a protein such as an antibody, gold fine particles and the analyte-bindable substance are mixed in a suitable buffer solution at room temperature for 5 minutes or more. After the reaction, the precipitate yielded by centrifugation is dispersed in a solution containing a dispersant such as polyethylene glycol, whereby the desired gold fine particle-labeled analyte-bindable substance can be obtained. When gold colloid particles are used, commercially available ones may be used. Alternatively, gold colloid particles may be prepared by a common method, for example a method of reducing chloroauric acid with sodium citrate (Nature Phys. Sci., vol. 241, 20 (1973) etc.). Moreover, commercially available gold labeled-antibodies or the like that have been already labeled with gold may also be employed.

In the present invention, the analyte-bindable substance is detected through labeling with fine metal particles and further sensitization with use of a silver-containing compound and a reducing agent for silver ion. Specifically, silver ions supplied from a silver-containing compound such as an organic silver salt are brought into contact with a reducing agent for silver ion; as a result, the silver ions are reduced by the reducing agent to form silver particles, which deposit on the gold label as a core, whereby the gold label is enhanced to enable highly sensitive analysis of the analyte.

### 5. Amplification Solution Containing a Silver-Containing Compound and a Reducing Agent for Silver Ion

An amplification solution that can be used in the present invention is what is called a developing solution as described in publications common in the field of photographic chemistry (e.g. “Kaitei Shashin kagaku no kiso, Ginen shashin hen (Revised Basic Photographic Engineering, silver salt photography),” (the Society of Photographic Science and Technology of Japan, Colona Publishing Co., Ltd.); “Shashin no kagaku (Photographic Chemistry),” (Akira Sasaki, Shashin Kogyo Shuppan); “Saishin Shoho Handbook (Latest Formulation Handbook),” (Shinichi Kikuchi et al., Amiko Shuppan); etc.).

In the present invention, any type of amplification solution can be used, as long as it is what is called a physical developing solution, which comprises silver ions, and such silver ions in the solution act as a core of development and reduction is carried out using a metal colloid as a center.

### 6. Silver-Containing Compound

The silver-containing compound used in the present invention may be an organic silver salt, an inorganic silver salt, or a silver complex.

The organic silver salt used in the present invention is an organic compound containing a reducible silver ion. Any one of an organic silver salt, an inorganic silver salt and a silver complex may be used as a compound containing a reducible silver ion in the present invention. For example, a silver nitrate, a silver acetate, a silver lactate, a silver butyrate, etc. have been known.

In addition, such a compound may be a silver salt or a coordination compound that forms a metallic silver relatively stable for light, when it is heated to 50° C. in the presence of a reducing agent.

The organic silver salt used in the present invention may be a compound selected from the silver salts of an azole compound and the silver salts of a mercapto compound. Such an azole compound is preferably a nitrogen-containing heterocyclic compound, and more preferably a triazole compound and a tetrazole compound. The mercapto compound is a compound having at least one mercapto group or thione group in the molecule thereof.

The silver salt of the nitrogen-containing heterocyclic compound of the present invention is preferably the silver salt of a compound having an imino group. Typical compounds include, but are not limited to, the silver salt of 1,2,4-triazole, the silver salt of benzotriazole or a derivative thereof (for example, a methylbenzotriazole silver salt and a 5-chlorobenzotriazole silver salt), a 1H-tetrazole compound such as phenylmercaptotetrazole described in U.S. Pat. No. 4,220,709, and imidazole or an imidazole derivative described in U.S. Pat. No. 4,260,677. Among these types of silver salts, a benzotriazole derivative silver salt or a mixture of two or more silver salts is particularly preferable.

The silver salt of the nitrogen-containing heterocyclic compound used in the present invention is most preferably the silver salt of a benzotriazole derivative.

The compound having a mercapto group or a thione group of the present invention is preferably a heterocyclic compound having 5 or 6 atoms. In this case, at least one atom in the ring is a nitrogen atom, and other atoms are carbon, oxygen, or sulfur atoms. Examples of such a heterocyclic compound include triazoles, oxazoles, thiazoles, thiazolines, imidazoles, diazoles, pyridines, and triazines. However, examples are not limited thereto.

Typical examples of the silver salt of the compound having a mercapto group or a thione group include, but are not limited to, the silver salt of 3-mercapto-4-phenyl-1,2,4-triazole, the silver salt of 2-mercapto-benzimidazole, the silver salt of 2-mercapto-5-aminothiazole, the silver salt of mercaptotriazine, the silver salt of 2-mercaptobenzoxazole, and the silver salt of compounds described in U.S. Pat. No. 4,123,274.

As such a compound having a mercapto group or a thione group of the present invention, a compound that does not contain a hetero ring may also be used. As such a mercapto or thione derivative that does not contain a hetero ring, an aliphatic or aromatic hydrocarbon compound having total 10 or more carbon atoms is preferable.

Among such mercapto or thione derivatives that do no contain a hetero ring, useful compounds include, but are not limited to, the silver salt of thioglycolic acid (for example, the silver salt of S-alkylthioglycolic acid having an alkyl group containing 12 to 22 carbon atoms) and the silver salt of dithiocarboxylic acid (for example, the silver salt of dithioacetic acid and the silver salt of thioamide).

An organic compound having the silver salt of carboxylic acid is also preferably used. It is straight-chain carboxylic acid, for example. Specifically, carboxylic acid containing 6 to 22 carbon atoms is preferably used. In addition, the silver salt of aromatic carboxylic acid is also preferable. Examples of such aromatic carboxylic acid and other carboxylic acids include, but are not limited to, substituted or unsubstituted silver benzoate (for example, silver 3,5-dihydroxybenzoate, silver o-methylbenzoate, silver m-methylbenzoate, silver p-rnethylbeinzoate, silver 2,4-dichlorobenzoate, silver acetamide benzoate and silver p-phenylbenzoate), silver tannate, silver phthalate, silver terephthalate, silver salicylate, silver phenylacetate, and silver pyromellitate.

In the present invention, aliphatic acid silver containing a thioether group as described in U.S. Pat. No. 3,330,663 can also be preferably used. A soluble silver carboxylate having a hydrocarbon chain containing an ether bond or a thioether bond, or a soluble silver carboxylate having a sterically hindered substituent on an α-position (of the hydrocarbon group) or an ortho-position (of the aromatic group) can also be used. These silver carboxylates have an improved solubility in a coating solvent, which provides a coating material having little light scattering.

Such silver carboxylates are described in U.S. Pat. No. 5,491,059. All of the mixtures of the silver salts described therein can be used in the invention, as necessary.

The silver salt of sulfonate as described in U.S. Pat. No. 4,504,575 can also be used in the embodiment of the present invention.

Further, for example, the silver salt of acetylene described in U.S. Pat. Nos. 4,761,361 and 4,775,613 can also be used in the present invention. It can be provided as a core-shell type silver salt as described in U.S. Pat. No. 6,355,408. Such silver salt is composed of a core consisting of one or more silver salts and a shell consisting of one or more different silver salts.

In the present invention, another product useful as a non-photosensitive silver source is a silver dimer composite consisting of two different types of silver salts described in U.S. Pat. No. 6,472,131. Such a non-photosensitive silver dimer composite consists of two different types of silver salts. When the aforementioned two types of silver salts include a linear saturated hydrocarbon group as a silver ligand, a difference in the numbers of carbon atoms of the ligands is 6 or greater.

The organic silver salt is contained as silver generally in an amount of 0.001 to 0.2 mol/m², and preferably 0.01 to 0.05 mol/m², in terms of the silver amount.

The inorganic silver salt or the silver complex used in the present invention is a compound containing a reducible silver ion. Preferably, such an inorganic silver salt or a silver complex is an inorganic silver salt or a silver complex, which forms metallic silver relatively stable for light, when the salt or complex is heated to 50° C. or higher in the presence of a reducing agent.

Examples of the inorganic silver salt used in the present invention include: a silver halide (such as silver chloride, silver bromide, silver chlorobromide, silver iodide, silver chloroiodide, silver chloroiodobromide, and silver iodobromide); the silver salt of a silver thiosulfate (e.g. a sodium salt, a potassium salt, an ammonium salt, etc.); the silver salt of a silver thiocyanate (e.g. a sodium salt, a potassium salt, an ammonium salt, etc.); and the silver salt of a silver sulfite (e.g. a sodium salt, a potassium salt, an ammonium salt, etc.).

The inorganic silver salt used in the present invention is preferably a silver halide or silver nitrate.

A method for forming the particles of the silver halide used in the invention is well known in the photographic industry. For example, methods described in Research Disclosure No. 17029, June 1978, and U.S. Pat. No. 3,700,458 may be used. Specifically, such a silver halide may be prepared by adding a silver-supplying compound (for example, a silver nitrate) and a halogen-supplying compound to a solution of a gelatin or other polymers.

The particle size of the silver halide is preferably very small in order to reduce examination noise. Specifically, the size is preferably 0.20 μm or less, more preferably 0.10 μm or less, and even more preferably in the range of nanoparticles. The term “particle size” is used herein to mean a diameter of a circular image having the same area as the projected area of the silver halide particle (the projected area of the main plane in the case of a tabular particle).

A silver thiosulfate, a silver thiocyanate, and a silver sulfite can also be prepared in the same manner as the formation of silver halide particles, by mixing a silver-supplying compound (such as a silver nitrate) with a thiosulfate (e.g. a sodium salt, a potassium salt, an ammonium salt, etc.), a thiocyanate (e.g. a sodium salt, a potassium salt, an ammonium salt, etc.), and a sulfite (e.g. a sodium salt, a potassium salt, an ammonium salt, etc.), respectively.

In general, if the concentration of silver ion in the amplification solution is too high, such silver ion is reduced in the amplification solution. In order to prevent such a phenomenon, a complexing agent may be used to cause the silver ion to form a complex. As such a complexing agent, amino acids such as glycine and histidine, heterocyclic bases, imidazole, benzimidazole, pyrazole, purine, pyridine, aminopyridine, nicotinamide, quinoline, and other similar aromatic heterocyclic compounds have been known. These compounds are described in E.P. Patent No. 0293947, for example. Further, as a complex salt-forming agent, thiosulfate, thiocyanate, and the like can also be used. Specific examples of the silver complex used in the present invention include a complex of a thiosulfate and a silver ion, a complex of a thiocyanate and a silver ion, a composite silver complex thereof, a complex of a sugar thione derivative and a silver ion, a complex of a cyclic imide compound (e.g. uracil, urazole, 5-methyluracil, barbituric acid, etc.) and a silver ion, and a complex of a 1,1-bissulfonylalkane and a silver ion. A preferred silver complex used in the invention is a complex of a cyclic imide compound (e.g. uracil, urazole, 5-methyluracil, barbituric acid, etc.) and a silver ion.

The silver complex used in the present invention may be prepared by a generally-known salt forming reaction. For example, the silver complex may be prepared by mixing in water or a water-miscible solvent a water-soluble silver supplier (such as a silver nitrate) with a ligand compound corresponding to the silver complex. The prepared silver complex can be used, after salts generated as by-products have been removed by a known desalting method such as dialysis or ultrafiltration.

The inorganic silver salt or the silver complex is contained as silver generally in an amount of 0.001 to 0.2 mol/m², and preferably 0.01 to 0.05 mol/m², in terms of the silver amount.

When an inorganic silver salt or a silver complex is used, a solvent for them is preferably used. The solvent used in the present invention is preferably a compound used as a ligand for forming a silver complex described in the above paragraphs for the “silver complex.” Examples of such a compound used as a solvent in the present invention include a thiosulfate, a thiocyanate, a sugar thione derivative, a cyclic imide compound, and a 1,1-bissulfonylalkane. The solvent used in the present invention is more preferably a cyclic imide compound such as uracil, urazole, 5-methyluracil, or barbituric acid. The solvent used in the present invention is preferably used at a molar ratio of 0.1 to 10 moles with respect to silver ions.

### 7. Reducing Agent Used for Silver Ion

As a reducing agent used for silver ion, either inorganic or organic materials capable of reducing silver(I) ion to silver, or the mixtures thereof, may be used.

As an inorganic reducing agent, reducible metal salts and reducible metal complex salts whose valence can be changed with metal ions such as Fe²⁺, V²⁺ or Ti³⁺ have been known. These salts can be used in the present invention. When such an inorganic reducing agent is used, it is necessary to form a complex with the oxidized ion or reduce it, so as to remove or detoxify the oxidized ion. For example, in a system using Fe⁺² as a reducing agent, citric acid or EDTA is used to form a complex with Fe³⁺ as an oxide, so as to detoxify it.

In the present system, such an inorganic reducing agent is preferably used. The metal salt of Fe²⁺ is more preferable.

Developing agents used for wet-process silver halide photographic-sensitized materials (for example, methyl gallate, hydroquinone, substituted hydroquinone, 3-pyrazolidones, p-aminophenols, p-phenylenediamines, hindered phenols, amidoximes, azines, catechols, pyrogallols, ascorbic acid (or derivatives thereof), and leuco dyes), or other materials known to those skilled in the art (see, for example, U.S. Pat. No. 6,020,117 (Bauer et al.)) may be used in the present invention.

The term “ascorbic acid reducing agent” means a complex of ascorbic acid and a derivative thereof. Ascorbic acid reducing agents are described in many publications, as described below, including, for example, U.S. Pat. No. 5,236,816 (Purol et al.) and publications cited therein.

The reducing agent used in the present invention is preferably an ascorbic acid reducing agent. Useful ascorbic acid reducing agents include ascorbic acid, an analogue thereof, an isomer thereof, and a derivative thereof. Examples of such compounds include the following compounds. However, examples are not limited thereto.

Examples of such compounds include D- or L-ascorbic acid and a sugar derivative thereof (for example, γ-lactoascorbic acid, glucoascorbic acid, fucoascorbic acid, glucoheptoascorbic acid, and maltoascorbic acid), sodium ascorbate, potassium ascorbate, isoascorbic acid (or L-erythroascorbic acid), and a salt thereof (for example, an alkali metal salt, an ammonium salt, or salts known in the art), and endiol-type ascorbic acid, enaminol-type ascorbic acid and thioenol-type ascorbic acid such as compounds described in U.S. Pat. No. 5,498,511, EP-A-0585,792, EP-A 0573700, EP-A 0588408, U.S. Pat. Nos. 5,089,819, 5,278,035, 5,384,232 and 5,376,510, JP 7-56286, U.S. Pat. No. 2,688,549, and Research Disclosure 37152 (March, 1995).

Among these compounds, D-, L-, and D,L-ascorbic acid (and an alkali metal salt thereof), and isoascorbic acid (and an alkali metal salt thereof) are preferable. Moreover, a sodium salt is a preferred salt thereof. If necessary, a mixture of these reducing agents may also be used.

A hindered phenol may be preferably used singly or in combination with one or more gradation-hardening reducing agents and/or contrast enhancers.

A hindered phenol is a compound having only one hydroxyl group on a benzene ring and also having at least one substituent at the ortho-position relative to the hydroxyl group. The hindered phenol reducing agent may have plural hydroxyl groups, as long as the hydroxyl groups are located on different benzene rings.

Examples of the hindered phenol reducing agent include binaphthols (that is, dihydroxybinaphthols), biphenols (that is, dihydroxybiphenols), bis(hydroxynaphthyl)methanes, bis(hydroxyphenyl)methanes (that is, bisphenols), hindered phenols, and hindered naphthols, each of which may be substituted.

Typical binaphthols include, but are not limited to, 1,1′-bi-2-naphthol, 1,1′-bi-4-methyl-2-naphthol, and compounds described in U.S. Pat. Nos. 3,094,417 and 5,262,295.

Typical biphenols include, but are not limited to, 2-(2-hydroxy-3 -t-butyl-5 -methylphenyl)-4-methyl-6-n-hexylphenol, 4,4′-dihydroxy-3,3 ′,5,5′-tetra-t-butylbiphenyl, 4,4′-dihydroxy-3,3′,5,5′-tetramethylbiphenyl, and compounds described in U.S. Pat. No. 5,262,295.

Typical bis(hydroxynaphthyl)methanes include, but are not limited to, 4,4′-methylenebis(2-methyl-1-naphthol) and compounds described in U.S. Pat. No. 5,262,295.

Typical bis(hydroxyphenyl)methanes include, but are not limited to, bis(2-hydroxy-3-t-butyl-5-methylphenyl)methane (CAO-5), 1,1 ′-bis(2-hydroxy-3,5-dimethylphenyl)-3,5,5-trimethyl hexane (NONOX or PERMANAX WSO), 1,1′-bis(3,5-di-t-butyl-4-hydroxyphenyl)methane, 2,2′-bis(4-hydroxy-3-methylphenyl) propane, 4,4 ′-ethylidene-bis(2-t-butyl-6-methylphenol), 2,2 ′-isobutylidene-bis(4,6-dimethylphenol) (LOWINOX 221B46), 2,2′-bis(3,5-dimethyl-4-hydroxyphenyl)propane, and compounds described in U.S. Pat. No. 5,262,295.

Typical hindered phenols include, but are not limited to, 2,6-di-t-butylphenol, 2,6-di-t-butyl-4-methylphenol, 2,4-di-t-butylphenol, 2,6-dichlorophenol, 2,6-dimethylphenol, and 2-t-butyl-6-methylphenol.

Typical hindered naphthols include, but are not limited to, 1-naphthol, 4-methyl-1-naphthol, 4-methoxy-1-naphthol, 4-chloro-1-naphthol, 2-methyl-1-naphthol, and compounds described in U.S. Pat. No. 5,262,295.

Moreover, other compounds disclosed as reducing agents include amidoximes (for example, phenylamidoxime), 2-thienylamidoxime, p-phenoxyphenylamidoxime, a combination of an aliphatic carboxylic allyl hydrazide and ascorbic acid (for example, a combination of 2,2′-bis(hydroxymethyl)-propionyl-p-phenyl hydrazide and ascorbic acid), a combination of a polyhydroxybenzene and at least one of hydroxylamine, reductone and hydrazine (for example, a combination of hydroquinone and bis(ethoxyethyl)hydroxylamine), piperidi-4-methylphenylhydrazine, hydroxamic acids (for example, phenylhydroxamic acid, p-hydroxyphenylhydroxamic acid, and o-alaninehydroxamic acid), a combination of an azine and a sulfonamidophenol (for example, a combination of phenothiazine and 2,6-dichloro-4-benzenesulfonamidophenol), α-cyanophenylacetic acid derivatives (for example, ethyl-α-cyano-2-methylphenylacetic acid and ethyl-α-cyanophenylacetic acid), bis-o-naphthol (for example, 2,2′-dihydroxy-1-binaphthyl, 6,6′-dibromo-2,2′-dihydroxy-1,1′-binaphthyl, and bis(2-hydroxy-1-naphthyl)methane), a combination of bis-naphthol and a 1,3-dihydroxybenzene derivative (for example, 2,4-dihydroxybenzophenone and 2,4-dihydroxyacetophenone), 5-pyrazolones (for example, 3-methyl-1-phenyl-5-pyrazolone), reductones (for example, dimethylaminohexose reductone, anhydrodihydro-aminohexose reductone, and anhydrodihydro-piperidone-hexose reductone), indane-1,3-diones (for example, 2-phenylindane-1,3-dione), chromans (for example, 2,2-dimethyl-7-t-butyl-6-hydroxychroman), 1,4-dihydroxypyridines (for example, 2,6-dimethoxy-3,5-dicarbetoxy-1,4-dihydropyridine), ascorbic acid derivatives (1-ascorbic palmitate, ascorbic stearate), unsaturated aldehydes (ketones), and 3-pyrazolidones.

Examples of a reducing agent that can be used in the present invention include substituted hydrazines such as sulfonyl hydrazines described in U.S. Pat. No. 5,464,738. Other useful reducing agents are described, for example, in U.S. Pat. Nos. 3,074,809, 3,094,417, 3,080,254 and 3,887,417. Auxiliary reducing agents described in U.S. Pat. No. 5,981,151 are also useful.

The reducing agent may be a combination of a hindered phenol reducing agent and a compound selected from various auxiliary reducing agents such as those mentioned below. In addition, a mixture of such a combined agent plus a contrast enhancer (that is, a mixture of the 3 components) is also useful. As such an auxiliary reducing agent, it is possible to use trityl hydrazide and formyl-phenyl hydrazide described in U.S. Pat. No. 5,496,695.

A contrast enhancer may be used in combination with the reducing agent. Useful contrast enhancers include, but are not limited to, hydroxylamines (including hydroxylamine and alkyl- and aryl-substituted derivatives thereof), alkanolamines and phthalic ammonium described in U.S. Pat. No. 5,545,505, hydroxamic acid compounds described in U.S. Pat. No. 5,545,507, N-acylhydrazine compounds described in U.S. Pat. No. 5,558,983, and hydrogen atom donor compounds described in U.S. Pat. No. 5,637,449.

Not all combinations of reducing agents and organic silver salts are equally effective. A preferred combination is a benzotriazole silver salt used as an organic silver salt, a substituted compound thereof or a mixture thereof, with an ascorbic acid reducing agent used as a reducing agent.

The reducing agent of the present invention may be contained in an amount of 1 weight % to 10 weight % (dry weight) based on the amount of silver in organic silver. When the reducing agent is added to a layer other than the layer containing the organic silver salt in a multilayer structure, the amount of the reducing agent is slightly higher, and it is desirably from approximately 2 weight % to approximately 15 weight %. An auxiliary reducing agent is contained in an amount of about 0.001 weight % to 1.5 weight % (dry weight).

### 8. Other Auxiliary Agents

Other auxiliary agents contained in the amplification solution may include a buffer, an antiseptic such as an antioxidant or an organic stabilizer, and a speed regulator. Examples of a buffer used herein include buffers comprising acetic acid, citric acid, sodium hydroxide, a salt thereof, or tris(hydroxymethyl)aminomethane, and other buffers used in ordinary chemical experiments. Using these buffers as appropriate, the pH of the amplification solution can be adjusted to the optimal pH.

The present invention will be more specifically described in the following examples. However, these examples are not intended to limit the scope of the present invention.

### EXAMPLES

### (I) Preparation of Silver Sensitization Solution

### (1) Preparation of Sensitization Solution A (Sensitization Solution of Example 1)

### (1-1) Preparation of Sensitization Solution A-1

In 325 g of water were dissolved 40 mL of 1 mol/L iron nitrate aqueous solution formed by dissolving iron (III) nitrate enneahydrate (Wako Pure Chemical Industries, 095-00995) in water, 10.5 g of citric acid (Wako Pure Chemical Industries, 038-06925), 0.1 g of dodecylamine (Wako Pure Chemical Industries, 123-00246), and 0.44 g of a surfactant C₉H₁₉—C₆H₄—O—(CH₂CH₂O)₅₀H. After all the ingredients were dissolved, 40 mL of nitric acid (10 weight %, Wako Pure Chemical Industries, 149-06845) was added thereto under stirring with a stirrer. 80 mL of this solution was measured out, to which 11.76 g of ammonium iron (II) sulfate hexahydrate (Wako Pure Chemical Industries, 091-00855) was added. The mixture was referred to as the sensitization solution A-1.

### (1-2) Preparation of Sensitization Solution A-2

Water was added to 10 mL of silver nitrate solution (containing 10 g of silver nitrate) to achieve the total weight of 100 g, by which the sensitization solution A-2 (10 weight % aqueous solution of silver nitrate) was prepared.

### (1-3) Preparation of Sensitization Solution A

40 mL of the sensitization solution A-1 was measured out, to which 4.25 mL of the sensitization solution A-2 was added. The mixture was stirred to obtain the sensitization solution A.

### (2) BBI Sensitization Solution (Sensitization Solution of Comparative Example 1)

The sensitization solution was prepared in accordance with the attached instruction manual of the Blotting Silver Enhancing Kit (BBI, SEKB250).

### (II) Evaluation of Detection Sensitivity

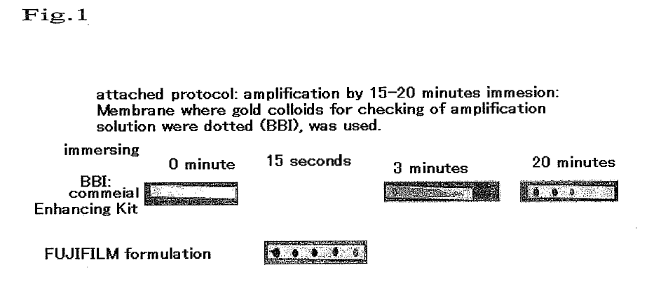

Sensitization test strips on which 10 ng, 1 ng, 100 pg, 10 pg, and 1 pg of gold labeled-proteins were respectively spotted, namely “Test Strip” (BBI, SETS10), were immersed in the sensitization solution (Example 1) prepared in the paragraph (1) in the abovementioned “(I) Preparation of Silver Sensitization Solution”, and the sensitization solution (Comparative Example 1) prepared in the paragraph (2) in the abovementioned “(I) Preparation of Silver Enhancer Solution” for a fixed time and then washed with water. The results are shown in FIG. 1. The degree of coloration was judged according to 4-point scale: darkly colored “+++”; colored “++”; slightly colored “+”; and uncolored “−” (Table 1).

### (III) Observation of Particle Shape After Sensitization

The backside of the sample was attached to the specimen stage with a carbon paste, followed by carbon shadowing. The surface of the sample was observed by SEM with FE-STEM S-5500 manufactured by Hitachi High Technologies, with reflection electrons at an acceleration voltage of 10 KV. Then, 100 signaling particles were selected, and the equivalent circular diameters thereof were measured using the projected areas of these particles, followed by calculation of the average value (Table 1).

TABLE 1

| | Example 1 | | Comparative Example 1 | | |
|---|---|---|---|---|---|
| | Pre- | 15 | Pre- | 3 | 20 |
| | sensitization | seconds | sensitization | minutes | minutes |
| Average | | 7.8 | | | 0.3 |
| particle | | | | | |
| diameter (μm) | | | | | |
| 10 ng | + | +++ | + | ++ | ++ |
| 1 ng | − | +++ | − | + | ++ |
| 100 pg | − | +++ | − | + | + |
| 10 pg | − | +++ | − | − | + |
| 1 pg | − | ++ | − | − | − |

### (Results)

Spots of 10 pg or higher concentration were only detectable after 20 minutes in Comparative Example 1 (the average particle diameter was 0.3 μm). On the other hand, in Example 1 (the average particle diameter was 7.8 μm), spots at lower concentration of 1 pg were sufficiently detectable after 15-second sensitization.

1. A blotting detection method which comprises moving an analyte held on a first carrier with a developing solution, and adsorbing the analyte onto a second carrier, wherein the analyte is labeled with metal fine particle and is detected by sensitization with use of a silver-containing compound and a reducing agent for silver ion, and the labeling substance having a size of not less than 1 μm and not more than 20 μm in the average particle size at the time of detection is detected.

2. The blotting detection method according to claim 1, wherein the second carrier is porous.

3. The blotting detection method according to claim 1, wherein the analyte is a protein or a nucleic acid.

4. The blotting detection method according to claim 1, wherein the metal fine particle is a gold fine particle.

5. The blotting detection method according to claim 1, wherein a time for the sensitization reaction with use of the silver-containing compound or the reducing agent for silver ion is within five minutes.

6. The blotting detection method according to claim 1, wherein a time for the sensitization reaction is within two minutes.

7. The blotting detection method according to claim 1, wherein the silver-containing compound is silver nitrate.

8. The blotting detection method according to claim 1, wherein the reducing agent for silver ion is Fe⁺².
